(19) United States
(12) Patent Application Publication
(10) Pub. No.: US 2024/0199631 A1
(43) Pub. Date: Jun. 20, 2024

(54) SYNTHESIS OF HUMAN SERUM ALBUMIN CONJUGATE

(71) Applicant: City of Hope, Duarte, CA (US)

(72) Inventors: Jun Xie, Duarte, CA (US); Yuelong Ma, Duarte, CA (US); David Horne, Duarte, CA (US)

(21) Appl. No.: 18/522,750

(22) Filed: Nov. 29, 2023

Related U.S. Application Data
(60) Provisional application No. 63/429,463, filed on Dec. 1, 2022.

(51) Int. Cl. C07D 491/22; A61K 47/64; C07K 14/765

(52) U.S. Cl. CPC C07D 491/22; A61K 47/643; C07K 14/765

(57) ABSTRACT

Described herein, inter alia, is a method of conjugating of human serum Albumin (HSA) to SN38.

### CROSS-REFERENCES TO RELATED APPLICATIONS

This application claims the benefit of priority under 35 U.S.C. § 119(e) of U.S. provisional application No. 63/429,463, filed Dec. 1, 2022, the disclosure of which is incorporated herein by reference in its entirety and for all purposes.

### BACKGROUND

SN38 (7-ethyl-10-hydroxy-camptothecin) is an active metabolite of irinotecan, an antineoplastic drug. The usage of SN38 is limited by its water solubility. Conjugation of SN38 with biomolecules provides possibility of improved water solubility and selective drug delivery.

Disclosed herein, inter alia, are solutions to these and other problems in the art.

### BRIEF SUMMARY

In an aspect, provided is a method of conjugating human serum albumin (HSA) to position 20 of SN-38. In embodiments, the method does not use chemical protection of position 10 prior to the conjugation reaction.

In an aspect, provided is a method of making succinyl-(C20—OH)SN38 comprising combining SN38, diazabicyclo[5.4.0]undec-7-ene (DBU) and succinic anhydride into a reaction vessel.

SN38 has the structure:

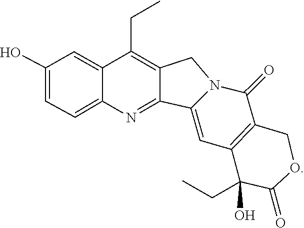 [SMILES: CCc1c2c(nc3ccc(O)cc13)-c1cc3c(c(=O)n1C2)COC(=O)[C@]3(O)CC]

Succinyl-(C20—OH)SN38 has the structure:

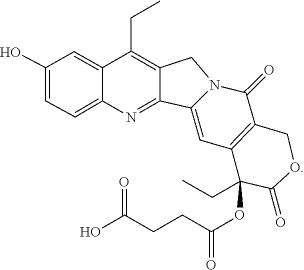 [SMILES: CCc1c2c(nc3ccc(O)cc13)-c1cc3c(c(=O)n1C2)COC(=O)[C@@]3(CC)OC(=O)CCC(=O)O]

In embodiments, the reaction vessel comprises dimethylformamide (DMF) as a solvent.

In embodiments, the reaction provides the yield of succinyl-(C20—OH)SN38 of greater than 65% to greater than 99%.

In embodiments, the method includes isolating the succinyl-(C20—OH)SN38 from the reaction mixture after producing succinyl-(C20—OH)SN38, thereby forming an isolated succinyl-(C20—OH)SN38. In embodiments, the succinyl-(C20—OH)SN38 is isolated from the reaction mixture as a precipitant.

Further provided is a method of isolating the succinyl-(C20—OH)SN38 from the reaction mixture after producing succinyl-(C20—OH)SN38, thereby forming an isolated succinyl-(C20—OH)SN38. Further provided is the method of isolating the succinyl-(C20—OH)SN38 from the reaction mixture as a precipitant.

In embodiments, the method further includes contacting the isolated succinyl-(C20—OH)SN38 with dicyclohexyl carbodiimide (DCC) in the presence of N-hydorxysuccinimide (NHS), thereby forming an activated succinyl-(C20—OH)SN38.

In embodiments, the method further includes contacting the activated succinyl-(C20—OH)SN38 with human serum albumin (HSA), wherein the HSA-SN38 conjugate has the formula:

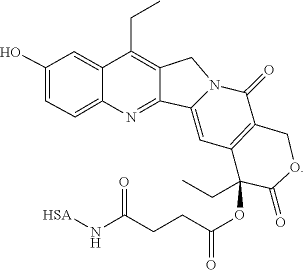

In embodiments, the method produces a population of HSA-SN38 conjugates. In embodiments, the population of the SN38-HSA conjugates includes a SN38-HSA conjugate having a SN38 to HSA ratio of at least one of 1:1, 2:1, 3:1, 4:1, 5:1, 6:1, 7:1, 9:1, 10:1, 11:1, 12:1, 13:1, 14:1, 15:1, 16:1, 17:1, 18:1, 19:1, or 20:1. In embodiments, the SN38 to HSA ratio is not 8:1.

In embodiments, the population comprises a SN38-HSA conjugate having a SN38-HSA ratio of at least one of 1:1, 2:1, 3:1, 4:1, 5:1, 6:1, 7:1, 8:1, 9:1, 10:1, 11:1, 12:1, 13:1, 14:1, 15:1, 16;1, 17:1, 18:1, 19:1, or 20:1.

In an aspect, provided is a population of HSA-SN38 conjugates. Further provided is a population of SN38-HSA conjugates, wherein the population comprises a SN38-HSA conjugate having a SN38-HSA ratio of at least one of 1:1, 2:1, 3:1, 4:1, 5:1, 6:1, 7:1, 8:1, 9:1, 10:1, 11:1, 12:1, 13:1, 14:1, 15:1, 16;1, 17:1, 18:1, 19:1, or 20:1

### BRIEF DESCRIPTION OF THE DRAWINGS

FIG. 1 depicts ¹H NMR of the purified SN38-succinic acid (SN38-SA).

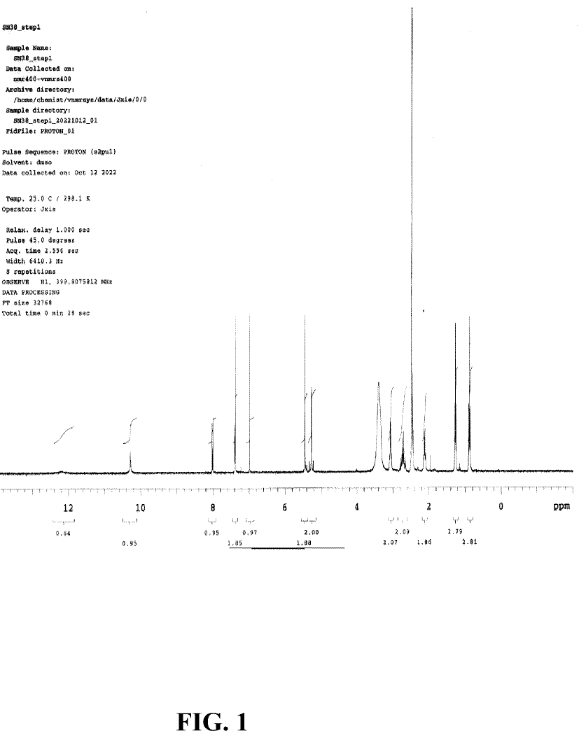

FIGS. 2A and 2B show ¹H NMR spectra of SN38-succinic acid (SN38-SA) for two characterized products at positions 10 (FIG. 2A) and position 20 (FIG. 2B). The data is taken from Int. J. Nanomedicine 10, 2295-2311, 2015.

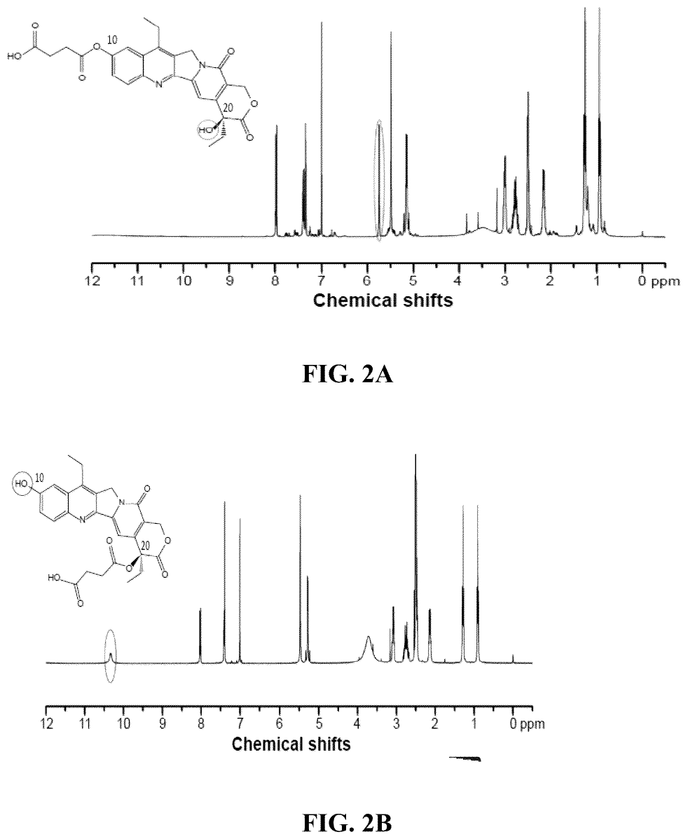

FIG. 3 depicts the comparison of the obtained NMR of SN38-SA based on method as disclosed in Mol Ther Oncolytics 12, 555-564, 2021, hereinafter “OSU” with literature NMR data as in FIG. 2B. The comparison revealed the OSU protocol yielded conjugated product as position 20, not at position 10, as originally reported in the OSU paper.

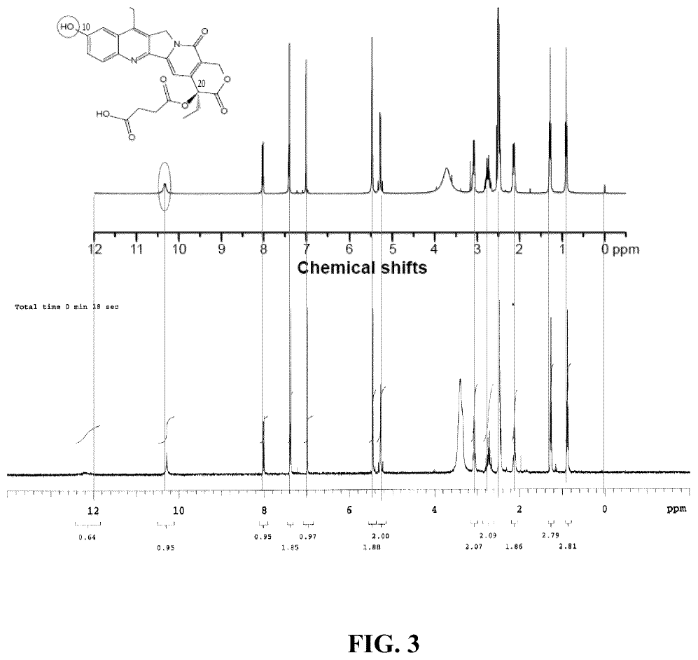

FIG. 4 shows that the comparison of NMR of the obtained product (lower panel) with the NMR of SN38-SA from literature (upper panel) is not consistent for conjugation at position 10.

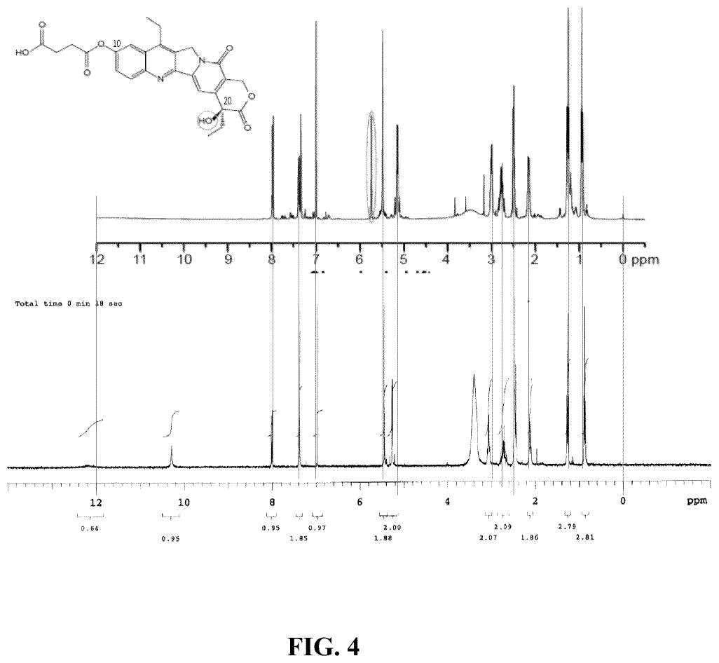

### DETAILED DESCRIPTION

I. Definitions

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵¹), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/- 10% of the specified value. In embodiments, about includes the specified value.

II. Methods

In an aspect, provided is a method of conjugating human serum albumin (HSA) to position 20 of SN-38. In embodiments, the method does not use chemical protection of position 10 prior to the conjugation reaction.

In an aspect, provided is a method of making succinyl-(C20—OH)SN38 comprising combining SN38, diazabicyclo[5.4.0]undec-7-ene (DBU) and succinic anhydride into a reaction vessel, thereby producing succinyl-(C20—OH)SN38. SN38 has the following structure:

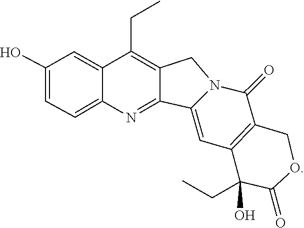 [SMILES: CCc1c2c(nc3ccc(O)cc13)-c1cc3c(c(=O)n1C2)COC(=O)[C@]3(O)CC]

Succinyl-(C20—OH)SN38 has the following structure:

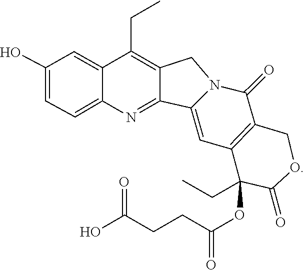 [SMILES: CCc1c2c(nc3ccc(O)cc13)-c1cc3c(c(=O)n1C2)COC(=O)[C@@]3(CC)OC(=O)CCC(=O)O]

In embodiments, the reaction vessel comprises dimethylformamide (DMF) as a solvent.

In embodiments, the method of making succinyl-(C20—OH)SN38 produces a yield of succinyl-(C20—OH)SN38 greater than 65% to greater than 99%. In embodiments, the method of making succinyl-(C20—OH)SN38 produces a yield of succinyl-(C20—OH)SN38 greater than 65%. In embodiments, the method of making succinyl-(C20—OH)SN38 produces a yield of succinyl-(C20—OH)SN38 greater than 70%. In embodiments, the method of making succinyl-(C20—OH)SN38 produces a yield of succinyl-(C20—OH)SN38 greater than 75%. In embodiments, the method of making succinyl-(C20—OH)SN38 produces a yield of succinyl-(C20—OH)SN38 greater than 80%. In embodiments, the method of making succinyl-(C20—OH)SN38 produces a yield of succinyl-(C20—OH)SN38 greater than 85%. In embodiments, the method of making succinyl-(C20—OH)SN38 produces a yield of succinyl-(C20—OH)SN38 greater than 90%. In embodiments, the method of making succinyl-(C20—OH)SN38 produces a yield of succinyl-(C20—OH)SN38 greater than 95%. In embodiments, the method of making succinyl-(C20—OH)SN38 produces a yield of succinyl-(C20—OH)SN38 greater than 98%. In embodiments, the method of making succinyl-(C20—OH)SN38 produces a yield of succinyl-(C20—OH)SN38 greater than 99%.

In embodiments, the method of making succinyl-(C20—OH)SN38 has a molar ratio of SN38 to DBU to succinic anhydride of 1 to 1.1 to 1.1. In embodiments, the method of making succinyl-(C20—OH)SN38 has a molar ratio of SN38 to DBU to succinic anhydride of 1 to at least 1.1 to at least 1.1. In embodiments, the method of making succinyl-(C20—OH)SN38 has a molar ratio of SN38 to DBU to succinic anhydride of 1 to 1.2 to 1:1. In embodiments, the method of making succinyl-(C20—OH)SN38 has a molar ratio of SN38 to DBU to succinic anhydride of 1 to at least 1.2 to at least 1:1. In embodiments, the method of making succinyl-(C20—OH)SN38 has a molar ratio of SN38 to DBU to succinic anhydride of 1 to 1.3 to 1:1. In embodiments, the method of making succinyl-(C20—OH)SN38 has a molar ratio of SN38 to DBU to succinic anhydride of 1 to at least 1.3 to at least 1:1. In embodiments, the method of making succinyl-(C20—OH)SN38 has a molar ratio of SN38 to DBU to succinic anhydride of 1 to 1.4 to 1:1. In embodiments, the method of making succinyl-(C20—OH)SN38 has a molar ratio of SN38 to DBU to succinic anhydride of 1 to at least 1.4 to at least 1:1. In embodiments, the method of making succinyl-(C20—OH)SN38 has a molar ratio of SN38 to DBU to succinic anhydride of 1 to 1.5 to 1:1. In embodiments, the method of making succinyl-(C20—OH)SN38 has a molar ratio of SN38 to DBU to succinic anhydride of 1 to at least 1.5 to at least 1:1. In embodiments, the method of making succinyl-(C20—OH)SN38 has a molar ratio of SN38 to DBU to succinic anhydride of 1 to 1.2 to 1:2. In embodiments, the method of making succinyl-(C20—OH)SN38 has a molar ratio of SN38 to DBU to succinic anhydride of 1 to at least 1.2 to at least 1:2. In embodiments, the method of making succinyl-(C20—OH)SN38 has a molar ratio of SN38 to DBU to succinic anhydride of 1 to 1.2 to 1:3. In embodiments, the method of making succinyl-(C20—OH)SN38 has a molar ratio of SN38 to DBU to succinic anhydride of 1 to at least 1.2 to at least 1:3. In embodiments, the method of making succinyl-(C20—OH)SN38 has a molar ratio of SN38 to DBU to succinic anhydride of 1 to 1.2 to 1:4. In embodiments, the method of making succinyl-(C20—OH)SN38 has a molar ratio of SN38 to DBU to succinic anhydride of 1 to at least 1.2 to at least 1:4. In embodiments, the method of making succinyl-(C20—OH)SN38 has a molar ratio of SN38 to DBU to succinic anhydride of 1 to 1.2 to 1:5. In embodiments, the method of making succinyl-(C20—OH)SN38 has a molar ratio of SN38 to DBU to succinic anhydride of 1 to at least 1.2 to at least 1:5. In embodiments, the method of making succinyl-(C20—OH)SN38 has a molar ratio of SN38 to DBU to succinic anhydride of 1 to 1.3 to 1:1. In embodiments, the method of making succinyl-(C20—OH)SN38 has a molar ratio of SN38 to DBU to succinic anhydride of 1 to at least 1.3 to at least 1:1. In embodiments, the method of making succinyl-(C20—OH)SN38 has a molar ratio of SN38 to DBU to succinic anhydride of 1 to 1.3 to 1:2. In embodiments, the method of making succinyl-(C20—OH)SN38 has a molar ratio of SN38 to DBU to succinic anhydride of 1 to at least 1.3 to at least 1:2. In embodiments, the method of making succinyl-(C20—OH)SN38 has a molar ratio of SN38 to DBU to succinic anhydride of 1 to 1.3 to 1:3. In embodiments, the method of making succinyl-(C20—OH)SN38 has a molar ratio of SN38 to DBU to succinic anhydride of 1 to at least 1.3 to at least 1:3. In embodiments, the method of making succinyl-(C20—OH)SN38 has a molar ratio of SN38 to DBU to succinic anhydride of 1 to 1.3 to 1:4. In embodiments, the method of making succinyl-(C20—OH)SN38 has a molar ratio of SN38 to DBU to succinic anhydride of 1 to at least 1.3 to at least 1:4. In embodiments, the method of making succinyl-(C20—OH)SN38 has a molar ratio of SN38 to DBU to succinic anhydride of 1 to 1.3 to 1:5. In embodiments, the method of making succinyl-(C20—OH)SN38 has a molar ratio of SN38 to DBU to succinic anhydride of 1 to at least 1.3 to at least 1:5. In embodiments, the method of making succinyl-(C20—OH)SN38 has a molar ratio of SN38 to DBU to succinic anhydride of 1 to 1.4 to 1:1. In embodiments, the method of making succinyl-(C20—OH)SN38 has a molar ratio of SN38 to DBU to succinic anhydride of 1 to at least 1.4 to at least 1:1. In embodiments, the method of making succinyl-(C20—OH)SN38 has a molar ratio of SN38 to DBU to succinic anhydride of 1 to 1.4 to 1:2. In embodiments, the method of making succinyl-(C20—OH)SN38 has a molar ratio of SN38 to DBU to succinic anhydride of 1 to at least 1.4 to at least 1:2. In embodiments, the method of making succinyl-(C20—OH)SN38 has a molar ratio of SN38 to DBU to succinic anhydride of 1 to 1.4 to 1:3. In embodiments, the method of making succinyl-(C20—OH)SN38 has a molar ratio of SN38 to DBU to succinic anhydride of 1 to at least 1.4 to at least 1:3. In embodiments, the method of making succinyl-(C20—OH)SN38 has a molar ratio of SN38 to DBU to succinic anhydride of 1 to 1.4 to 1:4. In embodiments, the method of making succinyl-(C20—OH)SN38 has a molar ratio of SN38 to DBU to succinic anhydride of 1 to at least 1.4 to at least 1:4. In embodiments, the method of making succinyl-(C20—OH)SN38 has a molar ratio of SN38 to DBU to succinic anhydride of 1 to 1.4 to 1:5. In embodiments, the method of making succinyl-(C20—OH)SN38 has a molar ratio of SN38 to DBU to succinic anhydride of 1 to at least 1.4 to at least 1:5. In embodiments, the method of making succinyl-(C20—OH)SN38 has a molar ratio of SN38 to DBU to succinic anhydride of 1 to 1.5 to 1:1. In embodiments, the method of making succinyl-(C20—OH)SN38 has a molar ratio of SN38 to DBU to succinic anhydride of 1 to at least 1.5 to at least 1:1. In embodiments, the method of making succinyl-(C20—OH)SN38 has a molar ratio of SN38 to DBU to succinic anhydride of 1 to 1.5 to 1:2. In embodiments, the method of making succinyl-(C20—OH)SN38 has a molar ratio of SN38 to DBU to succinic anhydride of 1 to at least 1.5 to at least 1:2. In embodiments, the method of making succinyl-(C20—OH)SN38 has a molar ratio of SN38 to DBU to succinic anhydride of 1 to 1.5 to 1:3. In embodiments, the method of making succinyl-(C20—OH)SN38 has a molar ratio of SN38 to DBU to succinic anhydride of 1 to at least 1.5 to at least 1:3. In embodiments, the method of making succinyl-(C20—OH)SN38 has a molar ratio of SN38 to DBU to succinic anhydride of 1 to 1.5 to 1:4. In embodiments, the method of making succinyl-(C20—OH)SN38 has a molar ratio of SN38 to DBU to succinic anhydride of 1 to at least 1.5 to at least 1:4. In embodiments, the method of making succinyl-(C20—OH)SN38 has a molar ratio of SN38 to DBU to succinic anhydride of 1 to 1.5 to 1:5. In embodiments, the method of making succinyl-(C20—OH)SN38 has a molar ratio of SN38 to DBU to succinic anhydride of 1 to at least 1.5 to at least 1:5.

In embodiments, the method includes isolating the succinyl-(C20—OH)SN38 from the reaction mixture after producing succinyl-(C20—OH)SN38, thereby forming an isolated succinyl-(C20—OH)SN38. In embodiments, the succinyl-(C20—OH)SN38 is isolated from the reaction mixture as a precipitant.

In embodiments, the method further includes contacting the isolated succinyl-(C20—OH)SN38 with dicyclohexyl carbodiimide (DCC) in the presence of N-hydorxysuccinimide (NHS), thereby forming an activated succinyl-(C20—OH)SN38.

In embodiments, the method further includes contacting the activated succinyl-(C20—OH)SN38 with human serum albumin (HSA), wherein the HSA-SN38 conjugate having the formula:

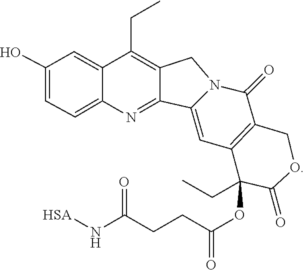

In embodiments, the method of making an SN38-HSA conjugate produces a population of SN38-HSA conjugates. Thus, in embodiments, a plurality of reactants are used in each step set forth above to produce a plurality of SN38-HSA conjugates, alternatively referred to herein as a population of SN38-HSA conjugates. In embodiments, the method of making an SN38-HSA conjugate produces a population of SN38-HSA conjugates including a SN38-HSA conjugate having a SN38 to HSA ratio of 1 to 1 to 25 to 1. In embodiments, the method of making an SN38-HSA conjugate produces a population of SN38-HSA conjugates including a SN38-HSA conjugate having a SN38 to HSA ratio of at least 1 to 1. In embodiments, the method of making an SN38-HSA conjugate produces a population of SN38-HSA conjugates including a SN38-HSA conjugate having a SN38 to HSA ratio of at least 2 to 1. In embodiments, the method of making an SN38-HSA conjugate produces a population of SN38-HSA conjugates including a SN38-HSA conjugate having a SN38 to HSA ratio of at least 3 to 1. In embodiments, the method of making an SN38-HSA conjugate produces a population of SN38-HSA conjugates including a SN38-HSA conjugate having a SN38 to HSA ratio of at least 4 to 1. In embodiments, the method of making an SN38-HSA conjugate produces a population of SN38-HSA conjugates including a SN38-HSA conjugate having a SN38 to HSA ratio of at least 5 to 1. In embodiments, the method of making an SN38-HSA conjugate produces a population of SN38-HSA conjugates including a SN38-HSA conjugate having a SN38 to HSA ratio of at least 6 to 1. In embodiments, the method of making an SN38-HSA conjugate produces a population of SN38-HSA conjugates including a SN38-HSA conjugate having a SN38 to HSA ratio of at least 7 to 1. In embodiments, the method of making an SN38-HSA conjugate produces a population of SN38-HSA conjugates including a SN38-HSA conjugate having a SN38 to HSA ratio of at least 8 to 1. In embodiments, the method of making an SN38-HSA conjugate produces a population of SN38-HSA conjugates including a SN38-HSA conjugate having a SN38 to HSA ratio of at least 9 to 1. In embodiments, the method of making an SN38-HSA conjugate produces a population of SN38-HSA conjugates including a SN38-HSA conjugate having a SN38 to HSA ratio of at least 10 to 1. In embodiments, the method of making an SN38-HSA conjugate produces a population of SN38-HSA conjugates including a SN38-HSA conjugate having a SN38 to HSA ratio of at least 11 to 1. In embodiments, the method of making an SN38-HSA conjugate produces a population of SN38-HSA conjugates including a SN38-HSA conjugate having a SN38 to HSA ratio of at least 12 to 1. In embodiments, the method of making an SN38-HSA conjugate produces a population of SN38-HSA conjugates including a SN38-HSA conjugate having a SN38 to HSA ratio of at least 13 to 1. In embodiments, the method of making an SN38-HSA conjugate produces a population of SN38-HSA conjugates including a SN38-HSA conjugate having a SN38 to HSA ratio of at least 14 to 1. In embodiments, the method of making an SN38-HSA conjugate produces a population of SN38-HSA conjugates including a SN38-HSA conjugate having a SN38 to HSA ratio of at least 15 to 1. In embodiments, the method of making an SN38-HSA conjugate produces a population of SN38-HSA conjugates including a SN38-HSA conjugate having a SN38 to HSA ratio of at least 16 to 1. In embodiments, the method of making an SN38-HSA conjugate produces a population of SN38-HSA conjugates including a SN38-HSA conjugate having a SN38 to HSA ratio of at least 17 to 1. In embodiments, the method of making an SN38-HSA conjugate produces a population of SN38-HSA conjugates including a SN38-HSA conjugate having a SN38 to HSA ratio of at least 18 to 1. In embodiments, the method of making an SN38-HSA conjugate produces a population of SN38-HSA conjugates including a SN38-HSA conjugate having a SN38 to HSA ratio of at least 19 to 1. In embodiments, the method of making an SN38-HSA conjugate produces a population of SN38-HSA conjugates including a SN38-HSA conjugate having a SN38 to HSA ratio of at least 20 to 1. In embodiments, the method of making an SN38-HSA conjugate produces a population of SN38-HSA conjugates including a SN38-HSA conjugate having a SN38 to HSA ratio of at least 21 to 1. In embodiments, the method of making an SN38-HSA conjugate produces a population of SN38-HSA conjugates including a SN38-HSA conjugate having a SN38 to HSA ratio of at least 22 to 1. In embodiments, the method of making an SN38-HSA conjugate produces a population of SN38-HSA conjugates including a SN38-HSA conjugate having a SN38 to HSA ratio of at least 23 to 1. In embodiments, the method of making an SN38-HSA conjugate produces a population of SN38-HSA conjugates including a SN38-HSA conjugate having a SN38 to HSA ratio of at least 24 to 1. In embodiments, the method of making an SN38-HSA conjugate produces a population of SN38-HSA conjugates including a SN38-HSA conjugate having a SN38 to HSA ratio of at least 25 to 1. In embodiments, the SN38 to HSA ratio is not 8:1.

In embodiments, the average ratio of SN38 to HSA in the population of HSA-SN38 conjugates is 1:1 to 2:1. In embodiments, the average ratio of SN38 to HSA in the population of HSA-SN38 conjugates is 2:1 to 3:1. In embodiments, the average ratio of SN38 to HSA in the population of HSA-SN38 conjugates is 3:1 to 4:1. In embodiments, the average ratio of SN38 to HSA in the population of HSA-SN38 conjugates is 4:1 to 5:1. In embodiments, the average ratio of SN38 to HSA in the population of HSA-SN38 conjugates is 5:1 to 6:1. In embodiments, the average ratio of SN38 to HSA in the population of HSA-SN38 conjugates is 6:1 to 7:1. In embodiments, the average ratio of SN38 to HSA in the population of HSA-SN38 conjugates is 7:1 to 8:1. In embodiments, the average ratio of SN38 to HSA in the population of HSA-SN38 conjugates is 8:1 to 9:1. In embodiments, the average ratio of SN38 to HSA in the population of HSA-SN38 conjugates is 9:1 to 10:1. In embodiments, the average ratio of SN38 to HSA in the population of HSA-SN38 conjugates is 10:1 to 11:1. In embodiments, the average ratio of SN38 to HSA in the population of HSA-SN38 conjugates is 11:1 to 12:1. In embodiments, the average ratio of SN38 to HSA in the population of HSA-SN38 conjugates is 12:1 to 13:1. In embodiments, the average ratio of SN38 to HSA in the population of HSA-SN38 conjugates is 13:1 to 14:1. In embodiments, the average ratio of SN38 to HSA in the population of HSA-SN38 conjugates is 14:1 to 15:1. In embodiments, the average ratio of SN38 to HSA in the population of HSA-SN38 conjugates is 15:1 to 16:1. In embodiments, the average ratio of SN38 to HSA in the population of HSA-SN38 conjugates is 16:1 to 17:1. In embodiments, the average ratio of SN38 to HSA in the population of HSA-SN38 conjugates is 17:1 to 18:1. In embodiments, the average ratio of SN38 to HSA in the population of HSA-SN38 conjugates is 18:1 to 19:1. In embodiments, the average ratio of SN38 to HSA in the population of HSA-SN38 conjugates is 19:1 to 20:1. In embodiments, the average ratio of SN38 to HSA in the population of HSA-SN38 conjugates is 20:1 to 21:1. In embodiments, the average ratio of SN38 to HSA in the population of HSA-SN38 conjugates is 21:1 to 22:1. In embodiments, the average ratio of SN38 to HSA in the population of HSA-SN38 conjugates is 22:1 to 23:1. In embodiments, the average ratio of SN38 to HSA in the population of HSA-SN38 conjugates is 23:1 to 24:1. In embodiments, the average ratio of SN38 to HSA in the population of HSA-SN38 conjugates is 24:1 to 25:1.

In embodiments the average ratio of SN38 to HSA in the population of HSA-SN38 conjugates is not 6:1 to 7:1. In embodiments the average ratio of SN38 to HSA in the population of HSA-SN38 conjugates is not 7:1 to 8:1. In embodiments the average ratio of SN38 to HSA in the population of HSA-SN38 conjugates is not 8:1 to 9:1.

In embodiments, the average ratio of SN38 to HSA in the population of HSA-SN38 conjugates (alternatively referred to herein as drug loading average) is calculated based on UV absorbance of SN38. In embodiments, the average ratio of SN38 to HSA in the population of HSA-SN38 conjugates is calculated based on relative mass abundance using a mass spectrometer.

In embodiments, the method produces a population of HSA-SN38 conjugates. In embodiments, the population of the SN38-HSA conjugates includes a SN38-HSA conjugate having a SN38 to HSA ratio of at least one of 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, or 25:1. In embodiments, the SN38 to HSA ratio is not 8:1. In embodiments, the population of the SN38-HSA conjugates includes a SN38-HSA conjugate having a SN38 to HSA ratio is 1:1, 2:1, 3:1, 4:1, 5:1, 6:1, 7:1, 9:1, 10:1, 11:1, 12: 1, 13:1, 14:1, 15:1, 16:1, 17:1, 18:1, 19:1, 20:1, 21: 1, 22:1, 23:1, 24:1, and/or 25:1, wherein the SN38 to HSA ratio is not 8:1.

In embodiments, the population comprises a SN38-HSA conjugate having a SN38-HSA ratio of at least one of 1:1, 2:1, 3:1, 4:1, 5:1, 6:1, 7:1, 8: 1, 9:1, 10:1, 11:1, 12:1, 13:1, 14:1, 15:1, 16:1, 17:1, 18:1, 19:1, 20:1, 21:1, 22:1, 23:1, 24:1, or 25:1. In embodiments, the SN38 to HSA ratio is not 8:1.

### NUMBERED EMBODIMENTS

Embodiment 1. A method of making succinyl-(C20—OH)SN38, said method comprising combining SN38, diazabicyclo[5.4.0]undec-7-ene) (DBU) and succinic anhydride into a reaction vessel, thereby producing succinyl-(C20—OH)SN38,
wherein SN38 has the structure:

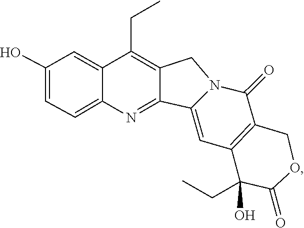 [SMILES: CCc1c2c(nc3ccc(O)cc13)-c1cc3c(c(=O)n1C2)COC(=O)[C@]3(O)CC]

and
wherein succinyl-(C20—OH)SN38 has the structure:

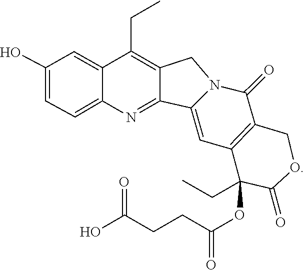 [SMILES: CCc1c2c(nc3ccc(O)cc13)-c1cc3c(c(=O)n1C2)COC(=O)[C@@]3(CC)OC(=O)CCC(=O)O]

Embodiment 2. The method of embodiment 1, wherein the reaction vessel comprises dimethylformamide (DMF) as a solvent.

Embodiment 3. The method of embodiment 1, wherein the yield of succinyl-(C20—OH)SN38 is greater than 65%.

Embodiment 4. The method of embodiment 3, wherein the yield of succinyl-(C20—OH)SN38 is greater than 80%.

Embodiment 5. The method of embodiment 4, wherein the yield of succinyl-(C20—OH)SN38 is greater than 90%.

Embodiment 6. The method of embodiment 1, wherein a molar ratio of SN38 to DBU to succinic anhydride is 1 to at least 1.1 to at least 1:1.

Embodiment 7. The method of embodiment 6, wherein the molar ratio of SN38 to DBU to succinic anhydride is 1 to at least 1.2 to at least 1:1.

Embodiment 8. The method of embodiment 6, wherein the molar ratio of SN38 to DBU to succinic anhydride is 1 to at least 1.3 to at least 1:1.

Embodiment 9. The method of embodiment 6, wherein the molar ratio of SN38 to DBU to succinic anhydride is 1 to at least 1.4 to at least 1:1.

Embodiment 10. The method of embodiment 6, wherein the molar ratio of SN38 to DBU to succinic anhydride is 1 to at least 1.5 to at least 1:1.

Embodiment 11. The method of embodiment 6, wherein the molar ratio of SN38 to DBU to succinic anhydride is 1 to at least 1.2 to at least 1:2.

Embodiment 12. The method of embodiment 6, wherein the molar ratio of SN38 to DBU to succinic anhydride is 1 to at least 1.2 to at least 1:3.

Embodiment 13. The method of embodiment 6, wherein the molar ratio of SN38 to DBU to succinic anhydride is 1 to at least 1.2 to at least 1:4.

Embodiment 14. The method of embodiment 6, wherein the molar ratio of SN38 to DBU to succinic anhydride is 1 to at least 1.2 to at least 1:5.

Embodiment 15. The method of embodiment 6, wherein the molar ratio of SN38 to DBU to succinic anhydride is 1 to at least 1.3 to at least 1:1.

Embodiment 16. The method of embodiment 6, wherein the molar ratio of SN38 to DBU to succinic anhydride is 1 to at least 1.3 to at least 1:2.

Embodiment 17. The method of embodiment 6, wherein the molar ratio of SN38to DBU to succinic anhydride is 1 to at least 1.3 to at least 1:3.

Embodiment 18. The method of embodiment 6, wherein the molar ratio of SN38 to DBU to succinic anhydride is 1 to at least 1.3 to at least 1:4.

Embodiment 19. The method of embodiment 6, wherein the molar ratio of SN38 to DBU to succinic anhydride is 1 to at least 1.3 to at least 1:5.

Embodiment 20. The method of embodiment 6, wherein the molar ratio of SN38 to DBU to succinic anhydride is 1 to at least 1.4 to at least 1:1.

Embodiment 21. The method of embodiment 6, wherein the molar ratio of SN38 to DBU to succinic anhydride is 1 to at least 1.4 to at least 1:2.

Embodiment 22. The method of embodiment 6, wherein the molar ratio of SN38 to DBU to succinic anhydride is 1 to at least 1.4 to at least 1:3.

Embodiment 23. The method of embodiment 6, wherein the molar ratio of SN38 to DBU to succinic anhydride is 1 to at least 1.4 to at least 1:4.

Embodiment 24. The method of embodiment 6, wherein the molar ratio of SN38 to DBU to succinic anhydride is 1 to at least 1.4 to at least 1:5.

Embodiment 25. The method of embodiment 6, wherein the molar ratio of SN38 to DBU to succinic anhydride is 1 to at least 1.5 to at least 1:1.

Embodiment 26. The method of embodiment 6, wherein the molar ratio of SN38 to DBU to succinic anhydride is 1 to at least 1.5 to at least 1:2.

Embodiment 27. The method of embodiment 6, wherein the molar ratio of SN38 to DBU to succinic anhydride is 1 to at least 1.5 to at least 1:3.

Embodiment 28. The method of embodiment 6, wherein the molar ratio of SN38 to DBU to succinic anhydride is 1 to at least 1.5 or to at least 1:4

Embodiment 29. The method of embodiment 6, wherein the molar ratio of SN38 to DBU to succinic anhydride is 1 to at least 1.5 to at least 1:5.

Embodiment 30. The method of embodiment 1, further comprising isolating the succinyl-(C20—OH)SN38 from the reaction mixture after producing succinyl-(C20—OH)SN38, thereby forming an isolated succinyl-(C20—OH)SN38.

Embodiment 31. The method of embodiment 30, wherein the succinyl-(C20—OH)SN38 is isolated from the reaction mixture as a precipitant.

Embodiment 32. The method of embodiment 30 or 31, further comprising contacting the isolated succinyl-(C20—OH)SN38 with dicyclohexyl carbodiimide (DCC) in the presence of N-hydroxysuccinimide (NHS), thereby forming an activated succinyl-(C20—OH)SN38.

Embodiment 33. The method of embodiment 32, further comprising contacting the activated succinyl-(C20—OH)SN38 with human serum albumin (HSA), thereby forming an HSA-SN38 conjugate having the formula:

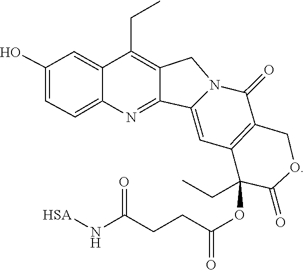

Embodiment 34. The method of embodiment 33, wherein the reaction produces a population of SN38-HSA conjugates, wherein the population comprises a SN38-HSA conjugate having a SN38 to HSA ratio of at least one of 1:1, 2:1, 3:1, 4:1, 5:1, 6:1, 7:1, 9:1, 10:1, 11:1 or 12:1.

Embodiment 35. The method of embodiment 34, wherein the population comprises a SN38-HSA conjugate having a SN38 to HSA ratio of at least 1:1.

Embodiment 36. The method of embodiment 34, wherein the population comprises a SN38-HSA conjugate having a SN38 to HSA ratio of at least 2:1.

Embodiment 37. The method of embodiment 34, wherein the population comprises a SN38-HSA conjugate having a SN38 to HSA ratio of at least 3:1.

Embodiment 38. The method of embodiment 34, wherein the population comprises a SN38-HSA conjugate having a SN38 to HSA ratio of at least 4:1.

Embodiment 39. The method of embodiment 34, wherein the population comprises a SN38-HSA conjugate having a SN38 to HSA ratio of at least 5:1.

Embodiment 40. The method of embodiment 34, wherein the population comprises a SN38-HSA conjugate having a SN38 to HSA ratio of at least 6:1.

Embodiment 41. The method of embodiment 34, wherein the population comprises a SN38-HSA conjugate having a SN38 to HSA ratio of at least 7:1.

Embodiment 42. The method of embodiment 34, wherein the population comprises a SN38-HSA conjugate having a SN38 to HSA ratio of at least 9:1.

Embodiment 43. The method of embodiment 34, wherein the population comprises a SN38-HSA conjugate having a SN38 to HSA ratio of at least 10:1.

Embodiment 44. The method of embodiment 34, wherein the population comprises a SN38-HSA conjugate having a SN38 to HSA ratio of at least 11:1.

Embodiment 45. The method of embodiment 34, wherein the population comprises a SN38-HSA conjugate having a SN38 to HSA ratio of at least 12:1.

Embodiment 46. A population of SN38-HSA conjugates, wherein the population comprises a SN38-HSA conjugate having a SN38 to HSA ratio of at least one of 1:1, 2:1, 3:1, 4:1, 5:1, 6:1, 7:1, 9:1, 10:1, 11:1 or 12:1.

Embodiment 47. The population of the SN38-HSA conjugates of claim 46, wherein a SN38 to HSA ratio is at least 1:1.

Embodiment 48. The population of the SN38-HSA conjugates of embodiment 46, wherein a SN38 to HSA ratio is at least 2:1.

Embodiment 49. The population of the SN38-HSA conjugates of embodiment 46, wherein a SN38 to HSA ratio is at least 3:1.

Embodiment 50. The population of the SN38-HSA conjugates of embodiment 46, wherein a SN38 to HSA ratio is at least 4:1.

Embodiment 51. The population of the SN38-HSA conjugates of embodiment 46, wherein a SN38 to HSA ratio is at least 5:1.

Embodiment 52. The population of the SN38-HSA conjugates of embodiment 46, wherein a SN38 to HSA ratio is at least 6:1.

Embodiment 53. The population of the SN38-HSA conjugates of embodiment 46, wherein a SN38 to HSA ratio is at least 7:1.

Embodiment 54. The population of the SN38-HSA conjugates of embodiment 46, wherein a SN38 to HSA ratio is at least 9:1.

Embodiment 55. The population of the SN38-HSA conjugates of embodiment 46, wherein a SN38 to HSA ratio is at least 10:1.

Embodiment 56. The population of the SN38-HSA conjugates of embodiment 46, wherein a SN38 to HSA ratio is at least 11:1.

Embodiment 57. The population of the SN38-HSA conjugates of embodiment 46, wherein a SN38 to HSA ratio is at least 12:1.

### EXAMPLES

### Example 1: Chemical Synthesis

Synthesis procedures were conducted using standard equipment and devices. Reactions were performed under inert atmosphere using nitrogen gas if not stated otherwise.

Sythesis of succinyl-(C20—OH)SN38

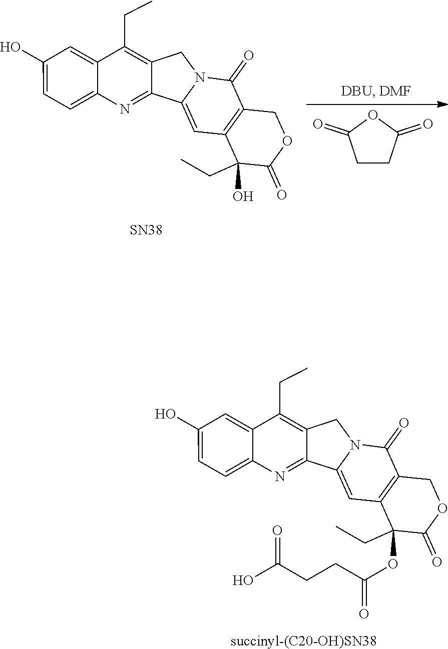

To a stirred suspension of SN38 (393 mg, 1 mmole) in 4 mL DMF was added succinic anhydride (150 mg, 1.5 mmole) and DBU (223 uL, 1.5 mmole) at rt. The reaction was stirred at rt for 3h and quenched with 0.1M HC1. The precipitation was filtered and washed with H2O. The product succinyl-(C20—OH)SN38 (400 mg, yield 81%) was obtained as yellow solid.

### Sythesis of SN38-HSA

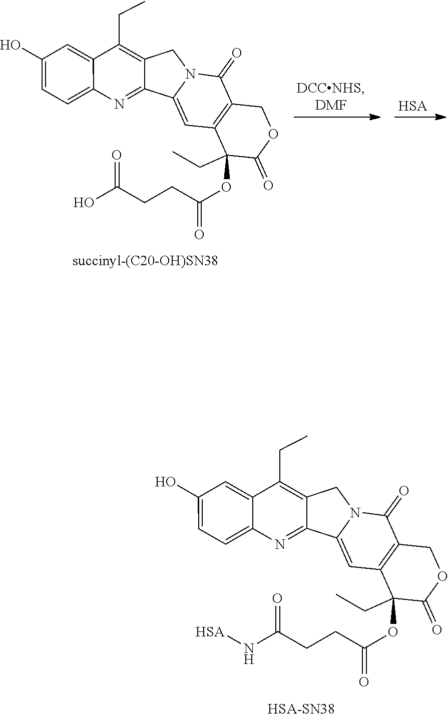

To a stirred solution of succinyl-(C20—OH) (8 mg, 16.2 umole) in DMF 80 uL was added NHS (N-hydroxysuccinimide) (2 mg, 17.4 umole) and DCC (3.6 mg, 17.4 umole) at rt. The reaction was stirred at rt for 16 h. Centrifuged at 10,000 g for 10 min to remove precipitation. The supernatant was added to 25% Human Serum Albumin (0.425 mL, 1.6 umole) diluted with 0.8 mL HEPES buffer (0.2 M, pH 8.5) and the mixture was stirred for 3 h at rt. Centrifuged at 10,000 g for 10 min to remove precipitation. Supernatant was desalted by PD-10 column and buffer exchanged into PBS to obtain HAS-SN38 (drug to protein ratio around 6:1). SN38 concentration was estimated by UV absorption at 382 or 360 nm and HSA concentration was measured by BCA assay.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of making succinyl-(C20—OH)SN38, said method comprising combining SN38, (diazabicyclo[5.4.0]undec-7-ene (DBU) and succinic anhydride into a reaction vessel, thereby producing succinyl-(C20—OH)SN38,
wherein SN38 has the structure: 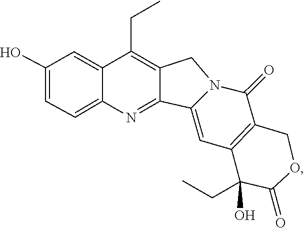 [SMILES: CCc1c2c(nc3ccc(O)cc13)-c1cc3c(c(=O)n1C2)COC(=O)[C@]3(O)CC]
and
wherein succinyl-(C20—OH)SN38 has the structure:

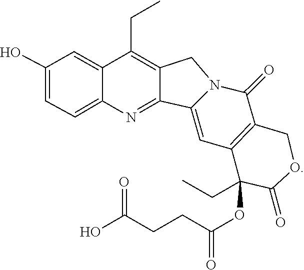 [SMILES: CCc1c2c(nc3ccc(O)cc13)-c1cc3c(c(=O)n1C2)COC(=O)[C@@]3(CC)OC(=O)CCC(=O)O]

2. The method of claim 1, wherein the reaction vessel comprises dimethylformamide (DMF) as a solvent.

3. The method of claim 2, wherein the yield of succinyl-(C20—OH)SN38 is greater than 65%.

4. The method of claim 3, wherein the yield of succinyl-(C20—OH)SN38 is greater than 80%.

5. The method of claim 7, wherein the yield of succinyl-(C20—OH)SN38 is greater than 90%.

6. The method of claim 1, wherein a molar ratio of SN38 to DBU to succinic anhydride is 1 to at least 1.1 to at least 1:1.

7-29. (canceled)

30. The method of claim 1, further comprising isolating the succinyl-(C20—OH)SN38 from the reaction mixture after producing succinyl-(C20—OH)SN38, thereby forming an isolated succinyl-(C20—OH)SN38.

31. The method of claim 30, wherein the succinyl-(C20—OH)SN38 is isolated from the reaction mixture as a precipitant.

32. The method of claim 30 er 31, further comprising contacting the isolated succinyl-(C20—OH)SN38 with dicyclohexyl carbodiimide (DCC) in the presence of N-hydroxysuccinimide (NHS), thereby forming an activated succinyl-(C20—OH)SN38.

33. The method of claim 32, further comprising contacting the activated succinyl-(C20—OH)SN38 with human serum albumin (HSA), thereby forming an HSA-SN38 conjugate having the formula:

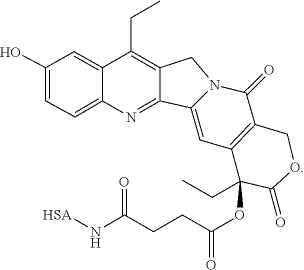

34. The method of claim 33, wherein the reaction produces a population of SN38-HSA conjugates, wherein the population comprises a SN38-HSA conjugate having a SN38 to HSA ratio of at least one of 1:1, 2:1, 3:1, 4:1, 5:1, 6:1, 7:1, 9:1, 10:1, 11:1 or 12:1.

35-45. (canceled)

46. A population of SN38-HSA conjugates, wherein the population comprises a SN38-HSA conjugate having a SN38 to HSA ratio of at least one of 1:1, 2:1, 3:1, 4:1, 5:1, 6:1, 7:1, 9:1, 10:1, 11:1 or 12:1.

47-57. (canceled)
